(19) United States
(12) Patent Application Publication
(10) Pub. No.: US 2024/0306982 A1
(43) Pub. Date: Sep. 19, 2024

(54) METHOD FOR TREATING SLEEP APNEA/HYPOPNEA

(71) Applicant: Laura SCHNEIDER, Loveland, CO (US)

(72) Inventor: Laura SCHNEIDER, Loveland, CO (US)

(21) Appl. No.: 18/122,356

(22) Filed: Mar. 16, 2023

(51) Int. Cl. A61B 5/00

(52) U.S. Cl. CPC A61B 5/4818

(57) ABSTRACT

A method of treating obstructive sleep apnea/hypopnea syndrome includes determining a patient is a candidate for treatment, wherein the determining includes (a) determining the patient has at least three respiratory events during one hour of sleep, the respiratory events selected from the following: apnea, hypopnea, gasping, choking, and (b) excluding non-respiratory sleep disorders and airway anomalies. The method includes performing submental liposuction on the patient, wherein the performing comprises removing at least 10 milliliters of fat.

### BACKGROUND

Embodiments herein are related to treating obstructive sleep apnea/hypopnea. As of the time of this writing, very few means of treating sleep apnea/hypopnea are available. Available treatments focus on treating apnea—not hypopnea—and include surgical removal of excess tissue or anomalies inside the airways, using a device, such as a nasal insert to stabilize and open the airway, and using a continuous positive airway pressure machine, such as a CPAP or BiPAP machine, during sleep.

Unfortunately, these treatments do not address one of the primary causes of obstructive sleep apnea. There thus remains a need for an effective treatment for obstructive sleep apnea and/or other new and useful innovations.

### SUMMARY

An exemplary method of treating obstructive sleep apnea/hypopnea syndrome includes determining a patient is a candidate for treatment and performing submental liposuction on the patient. The determining includes determining the patient has at least three respiratory events during one hour of sleep, the respiratory events selected from the following: apnea, hypopnea, gasping, choking. The determining includes excluding non-respiratory sleep disorders and airway anomalies. The performing includes removing at least 10 milliliters of fat.

### BRIEF DESCRIPTION OF THE DRAWINGS

FIG. 1 is a flowchart of an exemplary method.

### DETAILED DESCRIPTION

Obstructive sleep apnea hypopnea syndrome (OSAHS) is a disease in which the airway is blocked to varying degrees during sleep. Apnea is a full obstruction of the airway when a person is asleep. Hypopnea occurs when a person's breathing is reduced by 50 percent or more, but less than 100 percent. Both hypopnea and sleep apnea are serious medical conditions that can lead to other health complications. The symptoms of hypopnea may include excessive daytime sleepiness, loud snoring, insomnia, difficulty with memory and concentration, mood changes, fatigue, waking at night, and feeling short of breath.

Current treatments are very limited, expensive, and offer little relief. The first line of defense includes telling patients who are overweight or obese to lose weight to “possibly” treat OSAHS. Unfortunately, weight loss while suffering from OSAHS is nearly impossible, due to the excessive daytime sleepiness most patients experience.

Beyond the instruction to lose weight, physicians typically turn to prescribing a continuous positive airway pressure (CPAP or BiPAP) machine for the patient to use during sleep. This solution is also of little help in that it is expensive and requires a lifetime of use. CPAP and BiPAP machines are also known to introduce infection and reduce sleep due to noise.

In desperate bids for a night of sleep, patients are known to seek non-physician supervised treatment. Nasal inserts are used to expand the nasal passages. These inserts may provide limited relief for some, but they are known to introduce infection through the nasal passages if poorly maintained. Some patients find limited relief using elevated pillows. Elevated pillows, however, can introduce back and hip problems.

Of particular concern, OSAHS is chronically under-diagnosed in women to begin with. Applicant theorizes that women sleep “lighter” than men, and thus are more likely to wake prior to the typical symptoms of OSAHS being witnessed. Specifically, women are significantly more likely to awaken before a true apnea or hypopnea event even occurs, and possibly even before gasping or choking, such that current diagnostic methods that include sleep studies with multiple leads attached to the female patient become nearly valueless, because the female patient is less likely to enter a deep sleep.

There therefore remains a need to treat obstructive sleep apnea/hypopnea syndrome particularly for female patients.

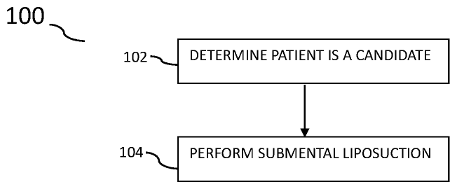

With reference to FIG. 1, a method of treating obstructive sleep apnea/hypopnea syndrome, method 100, is described.

The method 100 includes determining a patient is a candidate for treatment 100, wherein the determining includes: (a) determining the patient has at least three respiratory events during one hour of sleep, the respiratory events selected from the following: apnea, hypopnea, gasping, choking, and (b) excluding non-respiratory sleep disorders and airway anomalies.

The method 100 further includes performing submental liposuction on the patient 104. Submental liposuction may include removing at least 10 milliliters of fat, or at least 15 milliliters of fat, or at least 20 milliliters of fat, or at least 25 milliliters of fat, or at least 30 milliliters of fat, or at least 50 milliliters of fat.

In some embodiments, the determining 102 may include performing an endoscopic examination of the patient's airway, whereby airway anomalies are excluded. Airway anomalies may include cysts, tumors, skin flaps, or other features that do not typically form an airway passage.

In some embodiments, the method 100 may include filming the patient for at least one hour while the patient is asleep, wherein the filming comprises colleting video and audio recordings. In some embodiments, the filming may be performed in the absence of the use of electrodes. That is, the filming during sleep may be performed in a manner that more closely mimics a normal sleep night. The method 100 may include analyzing the video and audio recordings, whereby non-respiratory sleep disorders are excluded. The method 100 may include analyzing the video and audio recordings, whereby at least one of the following patient activities is witnessed: loud snoring, apnea, hypopnea, gasping, or choking.

The method 100 may include not affixing cables and/or cannulas to the patient during the determining the patient has at least three respiratory events during one hour of sleep. For example, the determining may be achieved through visual or audio means. Applicant suggests the use of visual or audio observation particularly for women, because women do not sleep as soundly as men, and are thus less likely to exhibit OSAHS during a sleep study that requires the use of cable leads, which are typically buckled or glued to the body to monitor the heart, while cannulas that monitor breathing actually interfere with breathing.

The method 100 may include determining the patient has a first oxygen saturation level, and determining the patient has a second oxygen saturation level less than the first oxygen saturation level during at least one of the at least three respiratory events. In some embodiments, the second oxygen saturation level is less than 90%.

The method 100 may include determining the patient is biologically female.

The method 100 described herein was tested and proved promising. A female patient with a BMI of 31 presented with excessive daytime drowsiness. She described episodes of awakening with a gasp and little sleep. An at-home sleep study determined the patient had 4.8 respiratory events in one hour, two of which were hypopnea. Her oxygen saturation level dropped to 88% multiple times. She snored 85% of the time she was asleep. Because she only had 4.8 episodes, the patient thus did not qualify for treatment in the United States.

Elevated pillows and nasal devices provided limited relief, if any. The patient then underwent tested submental liposuction as a treatment. Approximately 15 milliliters of fat were removed during the procedure.

The patient then underwent an in-lab sleep study. The in-lab sleep study determined she had 2 respiratory events during four hours of sleep, 0 minutes of snoring, and no significant drops in oxygen saturation level.

As a result, Applicant asserts that treating sleep apnea/hypopnea with submental liposuction is a viable option for some patients. This treats the disease and is less expensive, with permanent results, because weight re-gain in the chin area is very unlikely.

Each of the various elements disclosed herein may be achieved in a variety of manners. This disclosure should be understood to encompass each such variation, be it a variation of an embodiment of any apparatus embodiment, a method or process embodiment, or even merely a variation of any element of these. Particularly, it should be understood that the words for each element may be expressed by equivalent apparatus terms or method terms-even if only the function or result is the same. Such equivalent, broader, or even more generic terms should be considered to be encompassed in the description of each element or action. Such terms can be substituted where desired to make explicit the implicitly broad coverage to which this invention is entitled.

As but one example, it should be understood that all action may be expressed as a means for taking that action or as an element which causes that action. Similarly, each physical element disclosed should be understood to encompass a disclosure of the action which that physical element facilitates. Regarding this last aspect, the disclosure of a “attachment mechanism” should be understood to encompass disclosure of the act of “attaching”—whether explicitly discussed or not—and, conversely, were there only disclosure of the act of “attaching”, such a disclosure should be understood to encompass disclosure of a “attaching mechanism”. Such changes and alternative terms are to be understood to be explicitly included in the description.

Moreover, the claims shall be construed such that a claim that recites “at least one of A, B, or C” shall read on a device that requires “A” only. The claim shall also read on a device that requires “B” only. The claim shall also read on a device that requires “C” only. The claim shall also read on a device that requires “A+B”. The claim shall also read on a device that requires “A+B+C”, and so forth.

Those skilled in the art can readily recognize that numerous variations and substitutions may be made in the invention, its use and its configuration to achieve substantially the same results as achieved by the embodiments described herein.

Accordingly, there is no intention to limit the invention to the disclosed exemplary forms. Many variations, modifications and alternative constructions fall within the scope and spirit of the invention as expressed in the claims.

1. A method of treating obstructive sleep apnea/hypopnea syndrome, comprising:
determining a patient is a candidate for treatment, wherein the determining comprises
(a) determining the patient has at least three respiratory events during one hour of sleep, the respiratory events selected from the following: apnea, hypopnea, gasping, choking, and
(b) excluding non-respiratory sleep disorders and airway anomalies; and
performing submental liposuction on the patient, wherein the performing comprises removing at least 10 milliliters of fat.

2. The method of claim 1, wherein:
the determining comprises performing an endoscopic examination of the patient's airway, whereby airway anomalies are excluded.

3. The method of claim 1, wherein:
the performing submental liposuction comprises removing at least 15 milliliters of fat.

4. The method of claim 3, wherein:
the performing submental liposuction comprises removing at least 20 milliliters of fat.

5. The method of claim 4, wherein:
the performing submental liposuction comprises removing at least 25 milliliters of fat.

6. The method of claim 5, wherein:
the performing submental liposuction comprises removing at least 30 milliliters of fat.

7. The method of claim 6, wherein:
the performing submental liposuction comprises removing at least 50 milliliters of fat.

8. The method of claim 1, further comprising:
filming the patient for at least one hour while the patient is asleep, wherein the filming comprises colleting video and audio recordings.

9. The method of claim 8, further comprising:
analyzing the video and audio recordings, whereby non-respiratory sleep disorders are excluded.

10. The method of claim 8, further comprising:
analyzing the video and audio recordings, whereby at least one of the following patient activity is witnessed: loud snoring, apnea, hypopnea, gasping, or choking.

11. The method of claim 1, further comprising:
not affixing a cable to the patient during the determining the patient has at least three respiratory events during one hour of sleep.

12. The method of claim 1, further comprising:
not affixing a cannula to the patient during the determining the patient has at least three respiratory events during one hour of sleep.

13. The method of claim 1, further comprising:
determining the patient has a first oxygen saturation level;
determining the patient has a second oxygen saturation level less than the first oxygen saturation level during at least one of the at least three respiratory events.

14. The method of claim 13, wherein:
the second oxygen saturation level is less than 90%.

15. The method of claim 1, further comprising:
determining the patient is biologically female.
